SEQUENCE LISTING (6 sequences)

SEQ ID NO: 1 - protein, 19 aa, synthetic construct
CNPRPKNLDVGNKDGGSYD

SEQ ID NO: 2 - protein, 16 aa, synthetic construct
CHFGPYDNKARPNIPV

SEQ ID NO: 3 - protein, 125 aa, synthetic construct
EVKLQESGAELVKPGASVKMSCKASGYTFTSYWMHWVKQRPGQGLEWIGTIDPSDSYTSYNQKFKGKATLTVDTSSSTAYMQLSSLTSEDSAVYYCTRGEYGNAMDYWGQGTSVTVSSESQSFPN

SEQ ID NO: 4 - protein, 116 aa, synthetic construct
DIVLTQTPSYLAASPGETITINCRASKSISKYLAWYQEKPGKTNKLLIYSGSTLQSGIPSRFSGSGSGTDFTLTISSLEPEDFAMYYCQQHNEYPYTFGGGTKLEIKRADAAPTVS

SEQ ID NO: 5 - protein, 125 aa, synthetic construct
EVQLQESGPSLVKPSQTLSLTCSVTGDSITSGYWNWIRKFPGNKLDYMGYISYSGSTYYNPSLKSRISITRDTSKNQYYLQLNSVTTEDTATYYCARYRDVGFAYWGQGTLVTVSAAKTTPPSVY

SEQ ID NO: 6 - protein, 115 aa, synthetic construct
DIVLTQSPAIMSASPGEKVTMTCSASSSVSYMYWYQQKSGTSPKRWIYDTSKLASGVPARFSGSGSGTSYSLTISSMEAEDAATYYCQQWSSNPPTFGGGTKLEIKRADAAPTVS

(19) United States
(12) Patent Application Publication
(10) Pub. No.: US 2025/0250360 A1
(43) Pub. Date: Aug. 7, 2025

(54) ANTI-SULFATASE-RELATED ANTIGEN SPECIFIC ANTIBODY, DETECTION KIT COMPRISING ANTI-SULFATASE-RELATED ANTIGEN SPECIFIC ANTIBODY, AND METHOD FOR DETECTING OSTEOARTHRITIS AND PROGNOSTIC MONITORING OF OSTEOARTHRITIS PROGRESSION BY USING ANTI-SULFATASE-RELATED ANTIGEN SPECIFIC ANTIBODY

(71) Applicant: CCBiotech Inc., Hsinchu City (TW)

(72) Inventors: Ching-Chuan Jiang, Hsinchu City (TW); Chang-Hsun Hsieh, Hsinchu City (TW)

(21) Appl. No.: 19/025,053

(22) Filed: Jan. 16, 2025

Related U.S. Application Data
(60) Provisional application No. 63/549,588, filed on Feb. 5, 2024.

(51) Int. Cl. C07K 16/40; G01N 33/573

(52) U.S. Cl. CPC C07K 16/40; G01N 33/573; C07K 2317/565; G01N 2333/916; G01N 2800/105; G01N 2800/52

(57) ABSTRACT

The present disclosure provides an anti-sulfatase-related antigen specific antibody, a detection kit including the anti-sulfatase-related antigen specific antibody, and a method for detecting osteoarthritis by using the anti-sulfatase-related antigen specific antibody. The present disclosure achieves the effect of detecting osteoarthritis through various efficacy experiments.

### CROSS-REFERENCE TO RELATED APPLICATION

This application claims priority of Provisional application No. 63/549,588, filed on Feb. 5, 2024, the content of which is incorporated herein in its entirety by reference.

### STATEMENT REGARDING SEQUENCE LISTING

The sequence listing associated with this application is provided in text format in lieu of a paper copy and is hereby incorporated by reference into the specification. The name of the XML file containing the sequence listing is 113F0302_Sequence_listing. The XML file is 7000 bytes; was created on Dec. 31, 2024.

### BACKGROUND OF THE INVENTION

### 1. Field of the Invention

The present invention relates to an anti-sulfatase-related antigen specific antibody, a detection kit comprising the anti-sulfatase-related antigen specific antibody, and a method for detecting osteoarthritis (OA) and prognostic monitoring of osteoarthritis progression by using the anti-sulfatase-related antigen specific antibody. In particular, the anti-sulfatase-related antigen specific antibody of the present invention does not need to include sex and age as parameters for detecting osteoarthritis, and can achieve accurate detection results.

### 2. The Prior Art

Arthritis is one of the most common chronic diseases in the world. It is mainly caused by the deterioration of the cartilage of the joints or inflammation of the connective tissue, which causes joint pain and interferes with the normal movement of the joints. There are more than one hundred types in total. There are many causes of arthritis, and joint damage is also different. There are approximately 355 million arthritis patients in the world, of which more than 100 million are in China. In the United States, one in five people suffers from arthritis pain and discomfort; in Asia, one in six people suffer from the disease at some point in their lives. Arthritis is not a disease unique to the elderly, it can affect all ages, including children.

Arthritis can occur in the back, neck, knees, shoulder joints, hands, hip joints, and ankles. Most arthritis is related to human aging. People over sixty years old would suffer from some types of arthritis but young people would also suffer from arthritis. There are more than one hundred types of osteoarthritis (OA), rheumatoid, rheumatic and septic arthritis, traumatic osteoarthritis, and autoimmune arthritis, and ankylosing arthritis is also a type of arthritis.

Osteoarthritis is a symptom of diarthrosis failure. The main symptoms are degeneration of articular cartilage and friction between bones, resulting in joint pain, tenderness, stiffness, locking, joint effusion, decreased joint mobility, loss of joint space, osteophyte formation, cyst formation, joint deformation, and in severe cases may lead to disability. With the advent of an aging society, various chronic diseases have emerged one by one, and osteoarthritis has also become a serious problem. Osteoarthritis is a common joint disease in the world. 30% of the population in the United States is affected. The World Health Organization (WHO) has set the theme of bone and joint diseases from 2000 to 2010 since 2000. In the past ten years, October 12th is designated as “World Arthritis Day” every year. At present, nearly 400 million people in the world are suffering from arthritis. According to the GlobalData market report, the osteoarthritis market continues to grow from USD 1.6 billion in 2016 to 3.5 billion in 2026, with a compound annual growth rate (CAGR) of 8.1% from 2016 to 2026. According to the 2017 National Health Insurance Medical Statistics Annual Report, the expenditure on outpatient and hospitalization for osteoarthritis is nearly NT$5 billion, and the market potential is considerable. The current treatment for osteoarthritis is mainly pain management and cannot prevent further degeneration. When the pain seriously interferes with normal life and obvious functional deterioration, surgical operations such as total knee replacement (TKR) will be performed. Therefore, preventing the continuous degeneration of bones and joints and regenerating cartilage tissue is the ultimate goal of treating osteoarthritis.

Traditional methods of diagnosing osteoarthritis mainly rely on X-ray images. In addition, epidemiological studies have shown that the onset of degenerative arthritis is closely related to age and sex. Therefore, the traditional methods used to detect osteoarthritis need to include sex and age as parameters. However, using sex and age as parameters not only causes limitations and inconveniences in data collection, these factors may become confounding variables in clinical diagnosis, further increasing diagnostic uncertainty and risk of bias.

Current diagnostic tools, such as MRI or serum biomarker analysis, often face limitations in cost, accessibility, and the ability to detect early-stage osteoarthritis.

In view of the fact that the current methods for detecting osteoarthritis still have shortcomings of poor specificity and sensitivity. In order to solve the above-mentioned problems, those skilled in the art urgently need to develop a novel strategy for detecting osteoarthritis for the benefit of a large group of people in need thereof.

### SUMMARY OF THE INVENTION

A primary objective of the present invention is to provide an anti-sulfatase-related antigen specific antibody, which is selected from the group consisting of: an anti-sulfatase 1 (SULF1) specific antibody, comprising each amino acid sequence of SEQ ID NO:3 and SEQ ID NO:4; an anti-galactosamine (N-acetyl)-6-sulfatase (GALNs) specific antibody, comprising each amino acid sequence of SEQ ID NO:5 and SEQ ID NO:6; and a combination thereof.

Another objective of the present invention is to provide a detection kit, comprising the aforementioned anti-sulfatase-related antigen specific antibody.

Another objective of the present invention is to provide a method for detecting osteoarthritis (OA), comprising using the aforementioned anti-sulfatase-related antigen specific antibody.

Another objective of the present invention is to provide a method for prognostic monitoring of osteoarthritis progression, comprising using the aforementioned anti-sulfatase-related antigen specific antibody.

According to an embodiment of the present invention, each amino acid sequence of SEQ ID NO:3 and SEQ ID NO:5 is a heavy chain.

According to an embodiment of the present invention, each amino acid sequence of SEQ ID NO:4 and SEQ ID NO:6 is a light chain.

According to an embodiment of the present invention, the amino acid sequence of SEQ ID NO:3 comprises a complementarity determining region 1 (CDR1), a complementarity determining region 2 (CDR2), and a complementarity determining region 3 (CDR3), wherein the CDR1 comprises each amino acid sequence of residues 25-32 of SEQ ID NO:3, the CDR2 comprises each amino acid sequence of residues 45-61 of SEQ ID NO:3, and the CDR3 comprises each amino acid sequence of residues 99-106 of SEQ ID NO:3.

According to an embodiment of the present invention, the amino acid sequence of SEQ ID NO:4 comprises a complementarity determining region 1 (CDR1), a complementarity determining region 2 (CDR2), and a complementarity determining region 3 (CDR3), wherein the CDR1 comprises each amino acid sequence of residues 27-32 of SEQ ID NO:4, the CDR2 comprises each amino acid sequence of residues 44-55 of SEQ ID NO:4, and the CDR3 comprises each amino acid sequence of residues 91-96 of SEQ ID NO:4.

According to an embodiment of the present invention, the amino acid sequence of SEQ ID NO:5 comprises a complementarity determining region 1 (CDR1), a complementarity determining region 2 (CDR2), and a complementarity determining region 3 (CDR3), wherein the CDR1 comprises each amino acid sequence of residues 27-38 of SEQ ID NO:5, the CDR2 comprises each amino acid sequence of residues 51-66 of SEQ ID NO:5, and the CDR3 comprises each amino acid sequence of residues 110-130 of SEQ ID NO:5.

According to an embodiment of the present invention, the amino acid sequence of SEQ ID NO:6 comprises a complementarity determining region 1 (CDR1), a complementarity determining region 2 (CDR2), and a complementarity determining region 3 (CDR3), wherein the CDR1 comprises each amino acid sequence of residues 27-35 of SEQ ID NO:6, the CDR2 comprises each amino acid sequence of residues 50-60 of SEQ ID NO:6, and the CDR3 comprises each amino acid sequence of residues 97-107 of SEQ ID NO:6.

According to an embodiment of the present invention, the anti-sulfatase-related antigen specific antibody is utilized in direct and indirect immunoassays for detecting osteoarthritis biomarkers in urine, blood, saliva or synovial fluid.

According to an embodiment of the present invention, the detection kit further comprises reagents for both qualitative and quantitative assessments of osteoarthritis biomarkers, compatible with automated immunoassay systems.

The present invention is based on quantitative biomarker detection technology, which can provide objective data that is not interfered by sex and age, and accurately reflects the true physiological status of joint degeneration. This advantage significantly reduces the subjectivity and confusion that may arise in traditional diagnostic methods, providing clinicians with a more reliable basis for decision-making.

In summary, the present invention achieves the effect of detecting osteoarthritis (OA) through experiments illustrated in the following examples. In particular, the combination of the anti-sulfatase 1 (SULF1) specific antibody and the anti-galactosamine (N-acetyl)-6-sulfatase (GALNs) specific antibody of the present invention does not need to include sex and age as parameters for detecting osteoarthritis, and can achieve accurate detection results.

The present invention not only enables accurate osteoarthritis detection without relying on demographic parameters but also demonstrates scalability across various biological samples, including urine, blood, saliva and synovial fluid. Furthermore, the detection kit can be adapted to multiple diagnostic platforms, such as ELISA and immunoblotting, ensuring wide applicability in clinical and research settings.

The present invention addresses the shortcomings of prior art by providing a cost-effective, non-invasive, and highly specific diagnostic tool based on urinary biomarkers.

The embodiments of the present invention will be further described below. The following examples are used to illustrate the present invention and are not intended to limit the scope of the present invention. Anyone skilled in the art can make some changes and modifications without departing from the spirit and scope of the present invention. Therefore, the scope of the present invention shall be defined by the appended claims.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1A shows the amino acid positions corresponding to each domain of the anti-sulfatase 1 (SULF1) specific antibody and the A450 nm absorbance value measured when the anti-sulfatase 1 (SULF1) specific antibody binds to the antigen peptide. The upper table is the antibody heavy chain, and the lower table is the antibody light chain.

FIG. 1B shows specificity test results of the anti-sulfatase 1 (SULF1) specific antibody.

FIG. 2A shows the amino acid positions corresponding to each domain of the anti-galactosamine (N-acetyl)-6-sulfatase (GALNs) specific antibody and the A₄₅₀ nm absorbance value measured when the anti-galactosamine (N-acetyl)-6-sulfatase (GALNs) specific antibody binds to the antigen peptide. The upper table is the antibody heavy chain, and the lower table is the antibody light chain.

FIG. 2B shows specificity test results of the anti-galactosamine (N-acetyl)-6-sulfatase (GALNs) specific antibody.

FIG. 3 shows the result of a clinical trial for small-batch mass production testing of the anti-sulfatase 1 (SULF1) specific antibody, with sex and age as parameters.

FIG. 4 shows the result of a clinical trial for small-batch mass production testing of the anti-galactosamine (N-acetyl)-6-sulfatase (GALNs) specific antibody, with sex and age as parameters.

FIG. 5 shows the result of a clinical trial for small-batch mass production testing of the anti-sulfatase 1 (SULF1) specific antibody and the anti-galactosamine (N-acetyl)-6-sulfatase (GALNs) specific antibody (best-index).

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise stated in the context, “a”, “the” and similar terms used in the specification (especially in the following claims) should be understood as including singular and plural forms.

In this embodiment, the detection reagent kit design for antigen design includes: sulfatase 1 (SULF1), which may comprise the amino acid sequence of SEQ ID NO: 1; and galactosamine (N-acetyl)-6-sulfatase (GALNs), which may comprise the amino acid sequence of SEQ ID NO:2.

In this embodiment, the detection reagent kit design for antibody design includes: an anti-sulfatase 1 (SULF1) specific antibody, which may comprise each amino acid sequence of SEQ ID NO:3 and SEQ ID NO:4, wherein the amino acid sequence of SEQ ID NO:3 is a heavy chain, and the amino acid sequence of SEQ ID NO:4 is a light chain.

Description of the preparation of monoclonal antibodies is as follows. 1. Antigen preparation: the antigen is designed and produced. HPLC verifies that the antigen concentration reaches more than 95%. 2. Animal immunity: mice are used for immune reaction. Each antigen corresponds to five mice. A total of six immunizations are performed. The interval between each immunization is about 14 days. After the fourth to sixth boosting immunization, blood was collected, and indirect ELISA was performed to detect the antibody titer in the mouse serum. Finally, the two mice with the highest titer strength were used for subsequent fusion of spleen cells and myeloma cells. 3. Antibody screening and monoclonalization: indirect ELISA was used to screen two antigens to identify positive cell lines. The limiting dilution method was used to culture monoclonal cells for cloning and confirm the positivity. 4. Antibody sequencing: after screening positive individual cells, gene expression analysis was performed to obtain the antibody sequence. IgBLAST was used to confirm the source and specificity of antibody genes and complete the analysis and summary of antibody sequences. 5. Antibody production and purification: the screened monoclonal cell lines were used for large-scale production of monoclonal antibodies using ascites culture method. The collected antibodies were purified using a Protein A/G affinity chromatography column and replaced with PBS buffer. The purified antibody is tested for concentration and purity and is confirmed to meet the application requirements before being aliquoted and stored at −80° C. 6. Antibody application: detection and analysis of urine samples using indirect ELISA.

The instructions for specimen detection and verification are as follows. 1. Specimen collection. Experimental group: patients in the third and fourth stages of osteoarthritis, urine collected for testing. Control group: those with no symptoms of joint pain, and those who have been confirmed by X-ray photos to have no obvious symptoms of osteoarthritis, would undergo urine collection. 2. Biomarker expression testing. Monoclonal antibody detection targets: sulfatase 1 (SULF1) and galactosamine (N-acetyl)-6-sulfatase (GALNs). The indirect ELISA photometric detection system was used to detect the expression levels of SULF1 and GALNS in patient urine based on the specificity of monoclonal antibodies. 3. Statistical analysis: Statistical analysis methods were used to evaluate the difference in detection amounts of SULF1 and GALNS in the urine of the experimental group and the control group. ROC curve analysis of the detection system was conducted to establish a detection model.

The present invention aims to design and prepare monoclonal antibodies with high specificity and stability for target proteins SULF1 and GALNs. In the examples, a standardized experimental process was adopted.

Antigen design and preparation: antigen sequences were designed for two target proteins, SULF1 (SEQ ID NO:1) and GALNs (SEQ ID NO:2), respectively. The purity of the synthesized antigens was confirmed to be over 95% by HPLC analysis. The aliquoted antigens are stored at −80° C. to ensure stability for subsequent immunological experiments.

Animal immunization: Six-week-old female Balb/c mice were used for immune responses. Five mice were immunized with each antigen. For the first immunization, 100 μg of the antigen was mixed with complete Freund's adjuvant (CFA) and injected subcutaneously. Supplementary immunizations were then performed every 14 days, using incomplete Freund's adjuvant (IFA) as an auxiliary injection, with 100 μg of antigen injected each time. Mouse sera were collected from the fourth to sixth immunizations, and the antibody titers were measured by indirect ELISA. The two mice with the highest titers were selected for subsequent experiments.

Antibody screening and monoclonalization: splenocytes were isolated from the mice with the highest titers and fused with FO myeloma cell line (ATCC CRL-1646) using Hybri-MAXTM (P7306). The fused product was inoculated into HAT medium (DMEM, 2 mM L-glutamine, 1X HT supplement, 1X HAT medium supplement, 1% penicillin-streptomycin mixture and 10% fetal bovine serum) in a 96-well culture plate, cultured at 5% CO₂, 37° C. 7-10 days after fusion, indirect ELISA was used to screen the wells with specific antibodies in the supernatant, and positive wells were selected for cloning using the limiting dilution method. After cloning, a monoclonal cell line that stably secretes antibodies was selected to further verify its specificity for two antigens, SULF1 and GALNs. The selected cell lines were stored in liquid nitrogen to ensure cell stability and continued production capacity.

Antibody production and purification: the screened monoclonal cell lines were used for large-scale production of monoclonal antibodies using ascites culture method. The collected antibodies were purified using a Protein A/G affinity chromatography column and replaced with PBS buffer. The purified antibody is tested for concentration and purity and is confirmed to meet the application requirements before being aliquoted and stored at −80° C.

This embodiment is directed to two antibodies (anti-GALNS specific antibody and anti-SULF1 specific antibody) and analyzes their specificity to the target proteins under different concentrations of antigen. The indirect ELISA detection method was used to conduct antibody specificity analysis, compare the specificity performance of antibodies to different concentrations of antigens, and BSA was used as the background control group for evaluation. The antigen was coated on a 96-well ELISA plate, the test antibody and the HRP-labeled secondary antibody were added for reaction, and the absorbance value was measured using a 450 nm wavelength microplate reader.

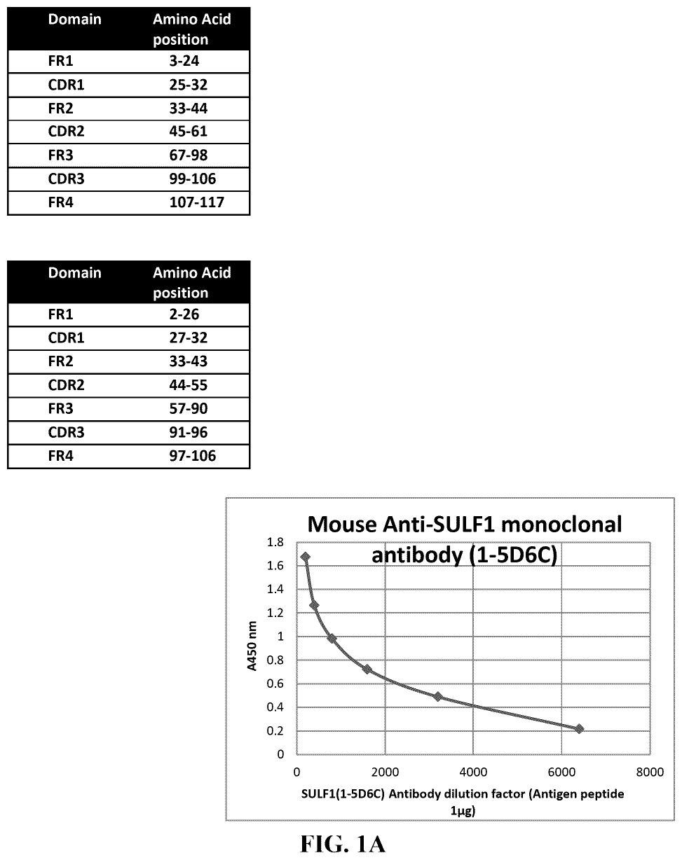

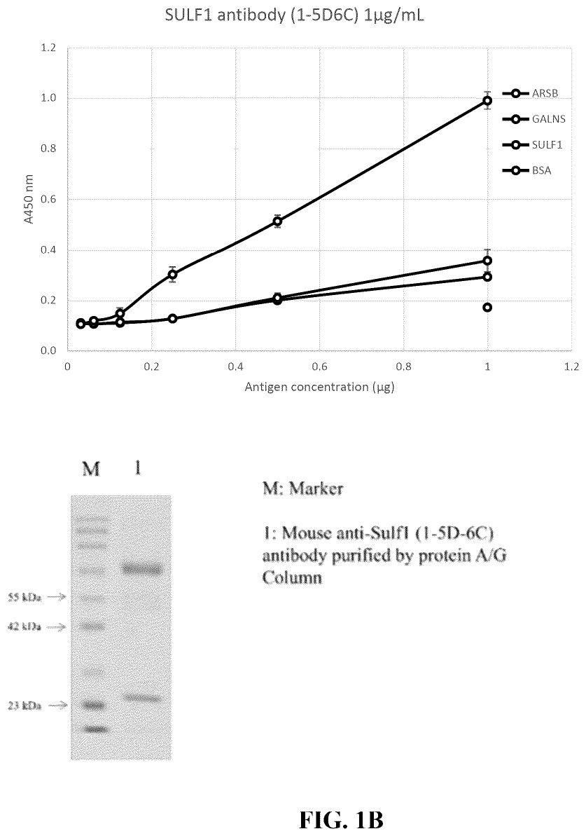

The amino acid positions corresponding to each domain of the anti-sulfatase 1 (SULF1) specific antibody and the A450 nm absorbance value measured when the anti-sulfatase 1 (SULF1) specific antibody binds to the antigen peptide are shown in FIG. 1A.

FIG. 1B shows specificity test results of the anti-sulfatase 1 (SULF1) specific antibody.

In this embodiment, the detection reagent kit design for antibody design includes: an anti-galactosamine (N-acetyl)-6-sulfatase (GALNs) specific antibody, which may comprise each amino acid sequence of SEQ ID NO:5 and SEQ ID NO:6, wherein the amino acid sequence of SEQ ID NO:5 is a heavy chain, and the amino acid sequence of SEQ ID NO:6 is a light chain.

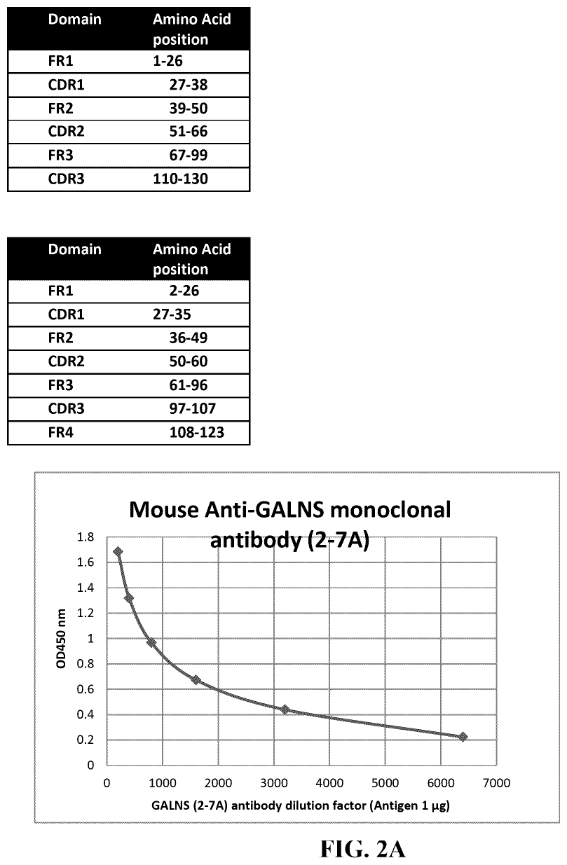

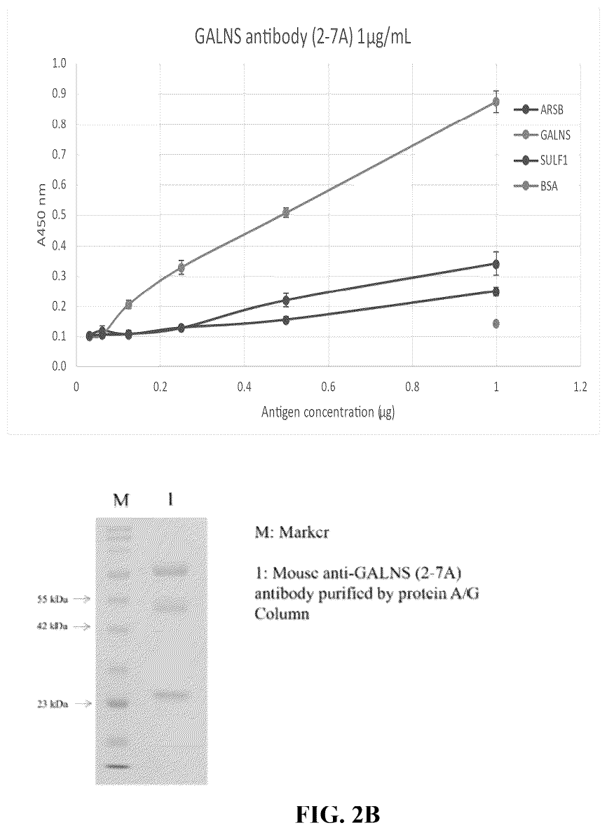

The amino acid positions corresponding to each domain of the anti-galactosamine (N-acetyl)-6-sulfatase (GALNs) specific antibody and the A₄₅₀ nm absorbance value measured when the anti-galactosamine (N-acetyl)-6-sulfatase (GALNs) specific antibody binds to the antigen peptide are shown in FIG. 2A.

FIG. 2B shows specificity test results of the anti-galactosamine (N-acetyl)-6-sulfatase (GALNs) specific antibody.

The anti-GALNS specific antibody shows good specificity at high concentration (1 μg/mL) of antigen, has a significantly higher response to the target antigen than other antigens, and can detect the low concentration antigen GALNS (125 ng/ml) to measure specific reaction. The anti-Sulf1 specific antibody has good specificity for SULF1 at high concentration (1 μg/mL) of antigen, with low cross-reactivity, and can detect low concentration antigen Sulf1 (250 ng/ml) to measure specificity reaction. Both antibodies can be used as potential tools for ELISA reagents for the diagnostic detection of different antigens.

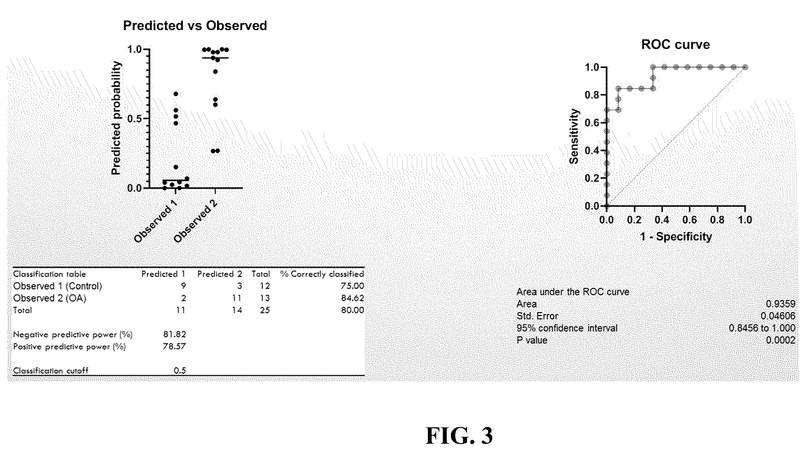

FIG. 3 shows the result of a clinical trial for small-batch mass production testing of the anti-sulfatase 1 (SULF1) specific antibody, with sex and age as parameters.

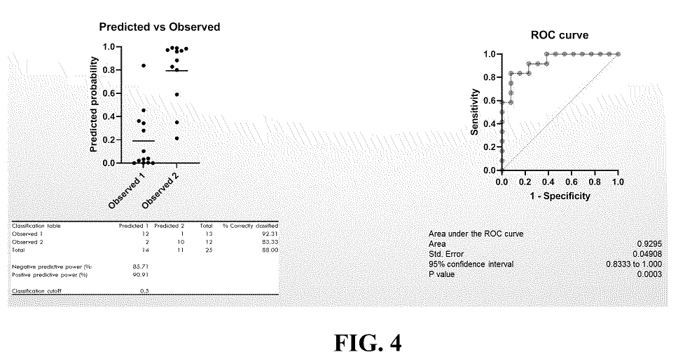

FIG. 4 shows the result of a clinical trial for small-batch mass production testing of the anti-galactosamine (N-acetyl)-6-sulfatase (GALNs) specific antibody, with sex and age as parameters.

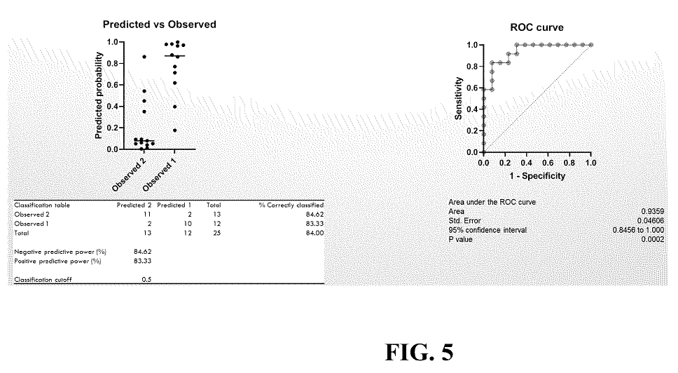

FIG. 5 shows the result of a clinical trial for small-batch mass production testing of the anti-sulfatase 1 (SULF1) specific antibody and the anti-galactosamine (N-acetyl)-6-sulfatase (GALNs) specific antibody (best-index). In particular, the combination of the anti-sulfatase 1 (SULF1) specific antibody and the anti-galactosamine (N-acetyl)-6-sulfatase (GALNs) specific antibody of the present invention does not need to include sex and age as parameters for detecting osteoarthritis, and can achieve accurate detection results.

As mentioned above, the onset of degenerative arthritis is closely related to age and sex. These factors may become confounding variables in clinical diagnosis, further increasing diagnostic uncertainty and risk of bias.

In summary, the present invention achieves the effect of detecting osteoarthritis (OA) through experiments illustrated in the above examples. Also, the present invention is based on quantitative biomarker detection technology, which can provide objective data that is not interfered by sex and age, and accurately reflects the true physiological status of joint degeneration. This advantage significantly reduces the subjectivity and confusion that may arise in traditional diagnostic methods, providing clinicians with a more reliable basis for decision-making.

The present invention not only enables accurate osteoarthritis detection without relying on demographic parameters but also demonstrates scalability across various biological samples, including urine, blood, saliva and synovial fluid. Furthermore, the detection kit can be adapted to multiple diagnostic platforms, such as ELISA and immunoblotting, ensuring wide applicability in clinical and research settings.

The present invention addresses the shortcomings of prior art by providing a cost-effective, non-invasive, and highly specific diagnostic tool based on urinary biomarkers.

Although the present invention has been described with reference to the preferred embodiments, it will be apparent to those skilled in the art that a variety of modifications and changes in form and detail may be made without departing from the scope of the present invention defined by the appended claims.

What is claimed is:

1. An anti-sulfatase-related antigen specific antibody, which is selected from the group consisting of:
an anti-sulfatase 1 (SULF1) specific antibody, comprising each amino acid sequence of SEQ ID NO:3 and SEQ ID NO:4;
an anti-galactosamine (N-acetyl)-6-sulfatase (GALNs) specific antibody, comprising each amino acid sequence of SEQ ID NO:5 and SEQ ID NO:6; and
a combination thereof.

2. The anti-sulfatase-related antigen specific antibody according to claim 1, wherein each amino acid sequence of SEQ ID NO:3 and SEQ ID NO:5 is a heavy chain.

3. The anti-sulfatase-related antigen specific antibody according to claim 1, wherein each amino acid sequence of SEQ ID NO:4 and SEQ ID NO:6 is a light chain.

4. The anti-sulfatase-related antigen specific antibody according to claim 2, wherein the amino acid sequence of SEQ ID NO:3 comprises a complementarity determining region 1 (CDR1), a complementarity determining region 2 (CDR2), and a complementarity determining region 3 (CDR3), wherein the CDR1 comprises each amino acid sequence of residues 25-32 of SEQ ID NO:3, the CDR2 comprises each amino acid sequence of residues 45-61 of SEQ ID NO:3, and the CDR3 comprises each amino acid sequence of residues 99-106 of SEQ ID NO:3.

5. The anti-sulfatase-related antigen specific antibody according to claim 3, wherein the amino acid sequence of SEQ ID NO:4 comprises a complementarity determining region 1 (CDR1), a complementarity determining region 2 (CDR2), and a complementarity determining region 3 (CDR3), wherein the CDR1 comprises each amino acid sequence of residues 27-32 of SEQ ID NO:4, the CDR2 comprises each amino acid sequence of residues 44-55 of SEQ ID NO:4, and the CDR3 comprises each amino acid sequence of residues 91-96 of SEQ ID NO:4.

6. The anti-sulfatase-related antigen specific antibody according to claim 2, wherein the amino acid sequence of SEQ ID NO:5 comprises a complementarity determining region 1 (CDR1), a complementarity determining region 2 (CDR2), and a complementarity determining region 3 (CDR3), wherein the CDR1 comprises each amino acid sequence of residues 27-38 of SEQ ID NO:5, the CDR2 comprises each amino acid sequence of residues 51-66 of SEQ ID NO:5, and the CDR3 comprises each amino acid sequence of residues 110-130 of SEQ ID NO:5.

7. The anti-sulfatase-related antigen specific antibody according to claim 3, wherein the amino acid sequence of SEQ ID NO:6 comprises a complementarity determining region 1 (CDR1), a complementarity determining region 2 (CDR2), and a complementarity determining region 3 (CDR3), wherein the CDR1 comprises each amino acid sequence of residues 27-35 of SEQ ID NO:6, the CDR2 comprises each amino acid sequence of residues 50-60 of SEQ ID NO:6, and the CDR3 comprises each amino acid sequence of residues 97-107 of SEQ ID NO:6.

8. The anti-sulfatase-related antigen specific antibody according to claim 1, which is utilized in direct and indirect immunoassays for detecting osteoarthritis biomarkers in urine, blood, saliva or synovial fluid.

9. A detection kit, comprising the anti-sulfatase-related antigen specific antibody according to claim 1.

10. The detection kit according to claim 9, wherein each amino acid sequence of SEQ ID NO:3 and SEQ ID NO:5 is a heavy chain.

11. The detection kit according to claim 9, wherein each amino acid sequence of SEQ ID NO:4 and SEQ ID NO:6 is a light chain.

12. The detection kit according to claim 10, wherein the amino acid sequence of SEQ ID NO:3 comprises a complementarity determining region 1 (CDR1), a complementarity determining region 2 (CDR2), and a complementarity determining region 3 (CDR3), wherein the CDR1 comprises each amino acid sequence of residues 25-32 of SEQ ID NO:3, the CDR2 comprises each amino acid sequence of residues 45-61 of SEQ ID NO:3, and the CDR3 comprises each amino acid sequence of residues 99-106 of SEQ ID NO:3.

13. The detection kit according to claim 11, wherein the amino acid sequence of SEQ ID NO:4 comprises a complementarity determining region 1 (CDR1), a complementarity determining region 2 (CDR2), and a complementarity determining region 3 (CDR3), wherein the CDR1 comprises each amino acid sequence of residues 27-32 of SEQ ID NO:4, the CDR2 comprises each amino acid sequence of residues 44-55 of SEQ ID NO:4, and the CDR3 comprises each amino acid sequence of residues 91-96 of SEQ ID NO:4.

14. The detection kit according to claim 10, wherein the amino acid sequence of SEQ ID NO:5 comprises a complementarity determining region 1 (CDR1), a complementarity determining region 2 (CDR2), and a complementarity determining region 3 (CDR3), wherein the CDR1 comprises each amino acid sequence of residues 27-38 of SEQ ID NO:5, the CDR2 comprises each amino acid sequence of residues 51-66 of SEQ ID NO:5, and the CDR3 comprises each amino acid sequence of residues 110-130 of SEQ ID NO:5.

15. The detection kit according to claim 11, wherein the amino acid sequence of SEQ ID NO:6 comprises a complementarity determining region 1 (CDR1), a complementarity determining region 2 (CDR2), and a complementarity determining region 3 (CDR3), wherein the CDR1 comprises each amino acid sequence of residues 27-35 of SEQ ID NO:6, the CDR2 comprises each amino acid sequence of residues 50-60 of SEQ ID NO:6, and the CDR3 comprises each amino acid sequence of residues 97-107 of SEQ ID NO:6.

16. The detection kit according to claim 9, wherein the anti-sulfatase-related antigen specific antibody is utilized in direct and indirect immunoassays for detecting osteoarthritis biomarkers in urine, blood, saliva or synovial fluid.

17. The detection kit according to claim 9, further comprising reagents for both qualitative and quantitative assessments of osteoarthritis biomarkers, compatible with automated immunoassay systems.

18. A method for detecting osteoarthritis (OA), comprising using the anti-sulfatase-related antigen specific antibody according to claim 1.

19. The method according to claim 18, wherein each amino acid sequence of SEQ ID NO:3 and SEQ ID NO:5 is a heavy chain.

20. The method according to claim 18, wherein each amino acid sequence of SEQ ID NO:4 and SEQ ID NO:6 is a light chain.

21. The method according to claim 19, wherein the amino acid sequence of SEQ ID NO:3 comprises a complementarity determining region 1 (CDR1), a complementarity determining region 2 (CDR2), and a complementarity determining region 3 (CDR3), wherein the CDR1 comprises each amino acid sequence of residues 25-32 of SEQ ID NO:3, the CDR2 comprises each amino acid sequence of residues 45-61 of SEQ ID NO:3, and the CDR3 comprises each amino acid sequence of residues 99-106 of SEQ ID NO:3.

22. The method according to claim 20, wherein the amino acid sequence of SEQ ID NO:4 comprises a complementarity determining region 1 (CDR1), a complementarity determining region 2 (CDR2), and a complementarity determining region 3 (CDR3), wherein the CDR1 comprises each amino acid sequence of residues 27-32 of SEQ ID NO:4, the CDR2 comprises each amino acid sequence of residues 44-55 of SEQ ID NO:4, and the CDR3 comprises each amino acid sequence of residues 91-96 of SEQ ID NO:4.

23. The method according to claim 19, wherein the amino acid sequence of SEQ ID NO:5 comprises a complementarity determining region 1 (CDR1), a complementarity determining region 2 (CDR2), and a complementarity determining region 3 (CDR3), wherein the CDR1 comprises each amino acid sequence of residues 27-38 of SEQ ID NO:5, the CDR2 comprises each amino acid sequence of residues 51-66 of SEQ ID NO:5, and the CDR3 comprises each amino acid sequence of residues 110-130 of SEQ ID NO:5.

24. The method according to claim 18, wherein the anti-sulfatase-related antigen specific antibody is utilized in direct and indirect immunoassays for detecting osteoarthritis biomarkers in urine, blood, saliva or synovial fluid.

25. A method for prognostic monitoring of osteoarthritis progression, comprising using the anti-sulfatase-related antigen specific antibody according to claim 1.
